(19) United States
(12) Patent Application Publication
(10) Pub. No.: US 2010/0028855 A1
(43) Pub. Date: Feb. 4, 2010

(54) DETECTION OF INFLUENZA VIRUS TYPE B

(75) Inventors: Peter S. Lu, Palo Alto, CA (US); Michael P. Belmares, San Jose, CA (US); Johannes Schweizer, Mountain View, CA (US); Jon Silver, San Jose, CA (US); Jon Silver, San Jose, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Arbor Vista Corporation, Sunnyvale, CA (US)

(21) Appl. No.: 12/298,133

(22) PCT Filed: Oct. 21, 2006
(86) PCT No.: PCT/US06/41748

§ 371 Date: Jun. 12, 2009

(51) Int. Cl. C12Q 1/70

(52) U.S. Cl. 435/5

(57) ABSTRACT

The invention provides methods for detecting influenza B from its NS1 protein. The NS1 protein is present in detectable levels in clinical samples and can be detected using antibodies that are panspecific for influenza B without binding to influenza A or other viruses.

### BACKGROUND OF THE INVENTION

Influenza is caused by an RNA virus of the orthomyxovilidae family. There are three types of these viruses and they cause three different types of influenza: type A, B and C. Influenza virus type A viruses infect mammals (humans, pigs, ferrets, horses) and birds. This is very important to mankind, as this is the type of virus that has caused worldwide pandemics. Influenza virus type B (also known simply as influenza B) infects only humans. It occasionally causes local outbreaks of flu. Influenza C viruses also infect only humans. They infect most people when they are young and rarely causes serious illness.

Current rapid immunodiagnostic tests for influenza antigens like “Binax NOW FluA and FluB™” (Binax, Inc., Portland, Me.), “Directigen Flu A+B™” (Becton Dickinson, Franklin Lakes, N.J.), “Flu OIA™” (Biostar Inc., Boulder, Colo.), “Quick Vue™” (Quidel, Sand Diego, Calif.), “Influ AB Quick™” (Denka Sieken Co., Ltd., Japan) and “Xpect Flu A & B” (Remel Inc., Lenexa, Kans.), can reportedly either detect influenza A or distinguish between Influenza A and B. The complexity of the test formats may require special training. In addition, significant amounts of virion particles are commonly required to obtain a positive test result, limiting their use to a short window of time when virus shedding is at its highest levels. Assay sensitivity is also variable with up to 20% false negative test results in certain assays being of significant current concern (e.g., see “WHO recommendations on the use of rapid testing for influenza diagnosis”, July 2005). Reverse-transcriptase PCR-based diagnostics (RT-PCR) has resulted in advances in capabilities, but is laborious and requires highly trained personnel making on-site or field-testing difficult. Because of the relative inefficiency of the reverse transcriptase enzyme, significant amounts of virus (e.g., 10⁴ virion particles) and as many as 20 primers may be required to effectively detect viral RNA. Unfortunately, RT-PCR is not easily adapted to high throughput screening of subjects in an epidemic setting or to field uses in an agricultural or point-of-care setting.

Additionally, the complexity, diversity and rapid emergence of new influenza strains has made diagnosis of high risk strains difficult, and therefore rapid response is at present nearly impossible. For epidemiologists, diversity resulting from high mutation rates and genetic reassortment make it difficult to anticipate where new strains may originate and respond with the timely introduction of new diagnostic primers for PCR. As a result, (at present) the diversity of influenza dictates the necessity of multiplex PCR approaches.

One of the present inventors has reported that the NS1 protein might exists in a different form in pathogenic forms of influenza A from typical non-avian forms of human influenza (i.e., influenza B or C), and may thus might be useful in identifying pathogenic forms of influenza A. Lu, Science 312, 337 (Apr. 21, 2006). Although infection by influenza B is not as serious as from influenza A, there remains a need to for improved methods to identify subjects infected with this virus both to distinguish them subjects infected with influenza and subjects suffering from other disorders presenting similar symptoms to influenza.

### BRIEF SUMMARY OF THE INVENTION

The invention provides methods for identifying whether a patient is infected with influenza virus type B. Such a method comprises determining whether NS1 protein of influenza virus type B is present in a patient sample, presence indicating the patient is infected with influenza virus type B. In some methods the determining comprises contacting a patient sample with an agent that specifically binds to influenza virus type B protein NS1; and detecting specific binding between the agent and the NS1 protein, specific binding indicating presence of the influenza virus type B. In some methods, the determining comprises determining the presence of mRNA encoding the NS1 protein, and inferring presence of the NS1 protein from the presence of the mRNA. Optionally, the agent is an antibody that specifically binds to the NS1 protein. Optionally, the antibody is panspecific for different strains of influenza type B. Optionally, the antibody is monospecific for a single strain of influenza type B. Optionally, the contacting step comprises, contacting the patient sample with first and second agents that specifically bind to different epitopes of influenza virus type B protein NS1, and the first agent is immobilized on a support, and the detecting step detects a sandwich in which the first and second agents are specifically bound to the NS1 protein to indicate presence of the virus. Optionally, the first and second agents are first and second antibodies. Optionally, the first and/or second agent is a polyclonal antibody. Optionally, the first and/or second agent is panspecific for different strains of influenza type B. In some methods, the patient sample is selected from the group consisting of blood, tissue, a nasal secretion, a lung exudate, a cloacal sample, a fecal sample, a throat swab and saliva.

Some methods further comprise determining whether the sample is infected with influenza virus type A. In some such methods, presence or absence of influenza virus type A is determined from presence or absence of influenza virus type A NS1 protein.

The invention further provides a kit for the identification and subtyping of influenza virus type B virus in a patient sample. Such a kit comprises an agent that specifically binds to the influenza virus type B NS1 protein, wherein said agent is immobilized on a solid support. Optionally, the agent is an antibody.

The invention further provides for the use of an NS1 protein of influenza virus type B to detect and/or quantify influenza virus type B.

The invention further provides for the use of an NS1 protein of influenza virus type C to detect and/or quantify influenza virus type C.

### BRIEF DESCRIPTION OF THE DRAWINGS

FIG. 1: Amino acid sequence of NS1 in two strains of influenza B.

FIGS. 2A and 1B show detection of recombinant NS1 from two strains of influenza B in an ELISA assay using various monoclonals to NS1 from influenza B. NS1 from several strains of influenza A are included as controls. None of the antibodies to NS1 from influenza B crossreacted with NS1 from influenza A.

FIG. 3: Detection of recombinant NS1 from two strains of influenza B in a lateral flow assay using various combinations of capture and detection antibody.

FIG. 4: Chart showing suitable combinations of capture and detection antibody for detection of NS1 from influenza B.

FIG. 5: Detection of NS1 from influenza B in clinical samples.

### DEFINITIONS

“Specific binding” between a binding agent, e.g., an antibody and an NS1 protein refers to the ability of a capture- or detection-agent to preferentially bind to a particular viral analyte that is present in a mixture of different viral analytes. For example, the antibodies described in the application specifically bind to NS1 from influenza B without specifically binding to NS1 from influenza A. Specific binding also means a dissociation constant (K_{D}) that is less than about 10⁻⁶ M; preferably, less than about 10⁻⁷M; and, most preferably, less than about 10⁻⁸ M

“Capture agent/analyte complex” is a complex that results from the specific binding of a capture agent, with an analyte, e.g. an influenza viral NS1 protein. A capture agent and an analyte specifically bind, i.e., the one to the other, under conditions suitable for specific binding, wherein such physicochemical conditions are conveniently expressed e.g. in terms of salt concentration, pH, detergent concentration, protein concentration, temperature and time. The subject conditions are suitable to allow binding to occur e.g. in a solution; or alternatively, where one of the binding members is immobilized on a solid phase. Representative conditions so-suitable are described in e.g., Harlow and Lane, “Antibodies: A Laboratory Manual”, Cold Spring Harbor Laboratory, Cold Spring Harbor, N.Y. (1989). Suitable conditions preferably result in binding interactions having dissociation constants (K_{D}) that are less than about 10⁻⁶M; preferably, less than about 10⁻⁷M; and, most preferably less than about 10⁻⁸M.

“Solid phase” means a surface to which one or more reactants may be attached electrostatically, hydrophobically, or covalently. Representative solid phases include e.g.: nylon 6; nylon 66; polystyrene; latex beads; magnetic beads; glass beads; polyethylene; polypropylene; polybutylene; butadiene-styrene copolymers; silastic rubber; polyesters; polyamides; cellulose and derivatives; acrylates; methacrylates; polyvinyl; vinyl chloride; polyvinyl chloride; polyvinyl fluoride; copolymers of polystyrene; silica gel; silica wafers glass; agarose; dextrans; liposomes; insoluble protein metals; and, nitrocellulose. Representative solid phases include those formed as beads, tubes, strips, disks, filter papers, plates and the like. Filters may serve to capture analyte e.g. as a filtrate, or act by entrapment, or act by covalently-binding. A solid phase capture reagent for distribution to a user may consist of a solid phase coated with a “capture reagent”, and packaged (e.g., under a nitrogen atmosphere) to preserve and/or maximize binding of the capture reagent to an influenza NS1 analyte in a biological sample.

Biological samples include tissue fluids, tissue sections, biological materials carried in the air or in water and collected there from e.g. by filtration, centrifugation and the like, e.g., for assessing bioterror threats and the like. Alternative biological samples can be taken from fetus or egg, egg yolk, and amniotic fluids. Representative biological fluids include urine, blood, plasma, serum, cerebrospinal fluid, semen, lung lavage fluid, feces, sputum, mucus, water carrying biological materials and the like. Alternatively, biological samples include nasopharyngeal or oropharyngeal swabs, nasal lavage fluid, tissue from trachea, lungs, air sacs, intestine, spleen, kidney, brain, liver and heart, sputum, mucus, water carrying biological materials, cloacal swabs, sputum, nasal and oral mucus, and the like. Representative biological samples also include foodstuffs, e.g., samples of meats, processed foods, poultry, swine and the like. Biological samples also include contaminated solutions (e.g., food processing solutions and the like), swab samples from out-patient sites, hospitals, clinics, food preparation facilities (e.g., restaurants, slaughter-houses, cold storage facilities, supermarket packaging and the like). Biological samples may also include in-situ tissues and bodily fluids (i.e., samples not collected for testing), e.g., the instant methods may be useful in detecting the presence or severity or viral infection in the eye e.g., using eye drops, test strips applied directly to the conjunctiva; or, the presence or extent of lung infection by e.g. placing an indicator capsule in the mouth or nasopharynx of the test subject. Alternatively, a swab or test strip can be placed in the mouth. The biological sample may be derived from any tissue, organ or group of cells of the subject. In some embodiments a scrape, biopsy, or lavage is obtained from a subject. Biological samples may include bodily fluids such as blood, urine, sputum, and oral fluid; and samples such as nasal washes, swabs or aspirates, tracheal aspirates, chancre swabs, and stool samples. Methods are known to those of skill in the art for the collection of biological specimens suitable for the detection of individual pathogens of interest, for example, nasopharyngeal specimens such as nasal swabs, washes or aspirates, or tracheal aspirates in the case of high risk influenza A viruses involved in respiratory disease, oral swabs and the like. Optionally, the biological sample may be suspended in an isotonic solution containing antibiotics such as penicillin, streptomycin, gentamycin, and mycostatin.

### DETAILED DESCRIPTION OF THE INVENTION

Detectable levels of the influenza NS1 protein of influenza virus B can be found in body secretions, such as nasal secretions. The invention provides antibodies to influenza B that are panreactive different strains of influenza B without specifically binding to influenza A. Such antibodies allow detection of presence of influenza B from the presence of its NS1 protein.

### I. Influenza Virus

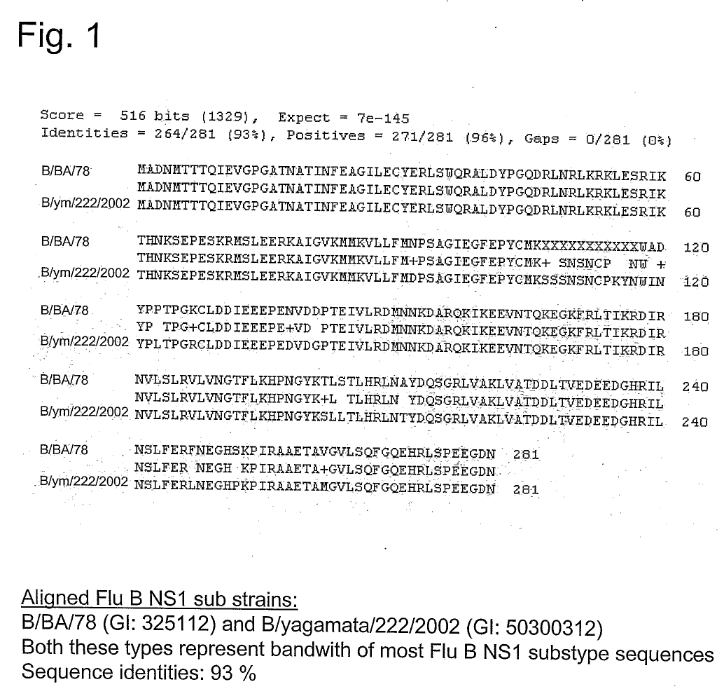

The influenza viruses belong to the Orthomyxoviridae family, and are classified into groups A, B, and C based upon antigenic differences in their nucleoprotein (NP) and matrix protein (M1). Further subtyping into strains is commonly based upon assessing the type of antigen present in two virion glycoproteins, namely, hemagglutinin (HA; H) and neuraminidase (NA; N). HA and NP are virulence factors mediating attachment of the virion to the surface of host cells. M1 protein is thought to function in virus assembly and budding, whereas NP functions in RNA replication and transcription. In addition to these virion proteins, two other non-structural, i.e., non-virion, proteins are expressed in virus infected cells which are referred to as non-structural proteins 1 and 2 (NS1; NS2). The non-structural viral protein NS1 has multiple functions including the regulation of splicing and nuclear export of cellular mRNAs and stimulation of translation, as well as the counteracting of host interferon ability. The NS1 protein has been identified and sequenced in influenza viruses and the sequence can be found in the NCBI database. The sequences of the NS1 protein from two exemplary strains of influenza B are shown in FIG. 1. The two strains show 93% sequence identity. The NS1 protein in other strains of influenza, means a protein having the greatest sequence similarity to one of the proteins identified as NS1 proteins in known influenza subtypes, using as sequence for example, the sequences shown in FIG. 1.

### II. Antibodies for Diagnostic and Therapeutic Uses

The invention provides antibodies to the NS1 protein of influenza B. Some such antibodies are panreactive in specifically binding to the NS1 strain from at least 2, or 5 or all or substantially all known strains of influenza B. Other antibodies are mono specific in specifically binding to only one strain of influenza B. Usually such antibodies lack specific binding to influenza A of all strains. The antibodies can be polyclonal antibodies, distinct monoclonal antibodies or pooled monoclonal antibodies with different epitope specificities. Monoclonal antibodies are made from antigen-containing fragments of the protein by standard procedures according to the type of antibody (see, e.g., Kohler, et al., Nature, 256:495, (1975); and Harlow & Lane, Antibodies, A Laboratory Manual (C.S.H.P., NY, 1988) Queen et al., Proc. Natl. Acad. Sci. USA 86:10029-10033 (1989) and WO 90/07861; Dower et al., WO 91/17271 and McCafferty et al., WO 92/01047 (each of which is incorporated by reference for all purposes).

Immunization can be biased to generate panspecific antibodies by immunizing with multiple strains of influenza B, or by immunizing with one strain and boosting with another. Alternatively, one can use a fragment from a highly conserved region of influenza B NS1 as the immunogen. Conversely, to generate a monospecific antibody, immunization with NS1 of a single strain, or a fragment of NS1 from a nonconserved region is preferred.

The term “antibody” or “immunoglobulin” is used to include intact antibodies and binding fragments thereof. Typically, fragments compete with the intact antibody from which they were derived for specific binding to an antigen fragment including separate heavy chains, light chains Fab, Fab′ F(ab′)2, Fabc, and Fv. Fragments are produced by recombinant DNA techniques, or by enzymatic or chemical separation of intact immunoglobulins. The term “antibody” also includes one or more immunoglobulin chains that are chemically conjugated to, or expressed as, fusion proteins with other proteins. The term “antibody” also includes bispecific antibody.

### III. Other Binding Agents

Although antibodies are preferred for use in detecting the NS1 protein, any binding agent with specific affinity for NS1 of influenza B can be used as an antibody surrogate. Surrogates includes peptides from randomized phage display libraries screened against NS1 from influenza B. Surrogates also include aptamers. Aptamers are RNA or DNA molecules selected in vitro from vast populations of random sequence that recognize specific ligands by forming binding pockets. Allosteric ribozymes are RNA enzymes whose activity is modulated by the binding of an effector molecule to an aptamer domain, which is located apart from the active site. These RNAs act as precision molecular switches that are controlled by the presence or absence of a specific effector. Aptamers can bind to nucleic acids, proteins, and even entire organisms. Aptamers are different from antibodies, yet they mimic properties of antibodies in a variety of diagnostic formats. Thus, aptamers can be used instead of or in combination with antibodies to identify the presence of general and specific NS1 regions.

### IV. Diagnostic Testing

Samples suspected of being infected with influenza B are tested for its presence by detecting the influenza virus B NS1 protein. The protein can be detected using antibodies or other capture reagents that specifically bind to the influenza B NS1 protein in formats described in more detail below. The presence of the influenza B NS1 protein signals that the sample is infected with influenza B virus. Such a test can be performed in isolation or in combination with other tests for influenza A and/or C. Testing for influenza A and influenza C can also be performed by detecting for presence of the NS1 protein of these strains using antibodies or other capture reagents with appropriate specificity for these strains. Methods of detecting influenza A and in particular distinguishing between pathogenic and nonpathogenic forms thereof are described in copending application Ser. No. 11/481,411 filed Jul. 3, 2006 (incorporated by reference in its entirety for all purposes).

The present methods are usually performed with antibodies or other binding reagents that are pan specific to NS1 of influenza B. The methods detect some or all strains of influenza without distinguishing between types. The methods can also be performed using antibodies that distinguish between strains of influenza B. In this case, usually a panel of antibodies is used in a single assay and the assay identifies not only presence of influenza B but which strain is present.

### V. Formats for Diagnostic Tests

The invention provides diagnostic capture and detect reagents useful in assay methods for identifying influenza B viruses in a variety of different types of biological samples. Such formats include immunoprecipitation, Western blotting, ELISA, radioimmunoassay, competitive and immunometric assays. See Harlow & Lane, Antibodies, A Laboratory Manual (CSHP NY, 1988); U.S. Pat. Nos. 3,791,932; 3,839,153; 3,850,752; 3,879,262; 4,034,074, 3,791,932; 3,817,837; 3,839,153; 3,850,752; 3,850,578; 3,853,987; 3,867,517; 3,879,262; 3,901,654; 3,935,074; 3,984,533; 3,996,345; 4,034,074; and 4,098,876.

Immunometric or sandwich assays are a preferred format (see U.S. Pat. Nos. 4,376,110, 4,486,530, 5,914,241, and 5,965,375). Such assays use one antibody or population of antibodies immobilized to a solid phase, and another antibody or population of antibodies in solution. Typically, the solution antibody or population of antibodies is labeled. If an antibody population is used, the population typically contains antibodies binding to different epitope specificities within the target antigen. Accordingly, the same population can be used for both solid phase and solution antibody. If monoclonal antibodies are used, first and second monoclonal antibodies having different binding specificities are used for the solid and solution phase. Solid phase and solution antibodies can be contacted with target antigen in either order or simultaneously. If the solid phase antibody is contacted first, the assay is referred to as being a forward assay. Conversely, if the solution antibody is contacted first, the assay is referred to as being a reverse assay. If target is contacted with both antibodies simultaneously, the assay is referred to as a simultaneous assay. After contacting the target with antibody, a sample is incubated for a period that usually varies from about 10 min to about 24 hr and is usually about 1 hr. A wash step can then be performed to remove components of the sample not specifically bound to the antibody(ies) being used as a detection reagent. When solid phase and solution antibodies are bound in separate steps, a wash can be performed after either or both binding steps. After washing, binding is quantified, typically by detecting label linked to the solid phase through binding of labeled solution antibody. Usually for a given pair of antibodies or populations of antibodies and given reaction conditions, a calibration curve is prepared from samples containing known concentrations of target antigen. Concentrations of antigen in samples being tested are then read by interpolation from the calibration curve. Analyte can be measured either from the amount of labeled solution antibody bound at equilibrium or by kinetic measurements of bound labeled solution antibody at a series of time points before equilibrium is reached. The slope of such a curve is a measure of the concentration of target in a sample.

Competitive assays can also be used. In some methods, target antigen in a sample competes with exogenously supplied labeled target antigen for binding to an antibody detection reagent. The amount of labeled target antigen bound to the antibody is inversely proportional to the amount of target antigen in the sample. The antibody can be immobilized to facilitate separation of the bound complex from the sample prior to detection (heterogeneous assays) or separation may be unnecessary as practiced in homogeneous assay formats. In other methods, the antibody used as a detection reagent is labeled. When the antibody is labeled, its binding sites compete for binding to the target antigen in the sample and an exogenously supplied form of the target antigen that can be, for example, the target antigen immobilized on a solid phase. Labeled antibody can also be used to detect antibodies in a sample that bind to the same target antigen as the labeled antibody in yet another competitive format. In each of the above formats, the antibody used as a detection reagent is present in limiting amounts roughly at the same concentration as the target that is being assayed.

Lateral flow devices are a preferred format. Similar to a home pregnancy test, lateral flow devices work by applying fluid to a test strip that has been treated with specific biologicals. Carried by the liquid sample, phosphors labeled with corresponding biologicals flow through the strip and can be captured as they pass into specific zones. The amount of phosphor signal found on the strip is proportional to the amount of the target analyte.

A sample suspected of containing influenza B is added to a lateral flow device, the sample is allowed to move by diffusion and a line or colored zone indicates the presence of Influenza B. The lateral flow typically contains a solid support (for example nitrocellulose membrane) that contains three specific areas: a sample addition area, a capture area containing one or more antibodies to NS1, and a read-out area that contains one or more zones, each zone containing one or more labels. The lateral flow can also include positive and negative controls. Thus, for example a lateral flow device can be used as follows: an influenza B NS1 protein is separated from other viral and cellular proteins in a biological sample by bringing an aliquot of the biological sample into contact with one end of a test strip, and then allowing the proteins to migrate on the test strip, e.g., by capillary action such as lateral flow. One or more antibodies, and/or aptamers are included as capture and/or detect reagents. Methods and devices for lateral flow separation, detection, and quantification are described by, e.g., U.S. Pat. Nos. 5,569,608; 6,297,020; and 6,403,383 incorporated herein by reference in their entirety. As an example, a test strip can comprise a proximal region for loading the sample (the sample-loading region) and a distal test region containing an antibody to an NS1 protein and buffer reagents and additives suitable for establishing binding interactions between the antibody any influenza B NS1 protein in the migrating biological sample. In another example, the test strip comprises two test regions that contain different antibodies to NS1 from two different strains of influenza B i.e., each is capable of specifically interacting with a different influenza B analyte.

Suitable detectable labels for use in the above methods include any moiety that is detectable by spectroscopic, photochemical, biochemical, immunochemical, electrical, optical, chemical, or other means. For example, suitable labels include biotin for staining with labeled streptavidin conjugate, fluorescent dyes (e.g., fluorescein, Texas red, rhodamine, green fluorescent protein, and the like), radiolabels (e.g., ³H, ¹²⁵I, ³⁵S, ¹⁴C, or ³²P), enzymes (e.g., horseradish peroxidase, alkaline phosphatase and others commonly used in an ELISA), and calorimetric labels such as colloidal gold or colored glass or plastic (e.g., polystyrene, polypropylene, latex beads). Patents that described the use of such labels include U.S. Pat. Nos. 3,817,837; 3,850,752; 3,939,350; 3,996,345; 4,277,437; 4,275,149; and 4,366,241. See also Handbook of Fluorescent Probes and Research Chemicals (6th Ed., Molecular Probes, Inc., Eugene Oreg.). Radiolabels can be detected using photographic film or scintillation counters, fluorescent markers can be detected using a photodetector to detect emitted light. Enzymatic labels are typically detected by providing the enzyme with a substrate and detecting the reaction product produced by the action of the enzyme on the substrate, and calorimetric labels are detected by simply visualizing the colored label.

The level of influenza B NS1 protein in a sample can be quantified and/or compared to controls. Suitable negative control samples are e.g. obtained from individuals lnown to be healthy, e.g., individuals known not to have an influenza viral infection. Specificity controls may be collected from individuals having known influenza A or influenza C infection, or individuals infected with viruses other than influenza. Control samples can be from individuals genetically related to the subject being tested, but can also be from genetically unrelated individuals. A suitable negative control sample can also be a sample collected from an individual at an earlier stage of infection, i.e., a time point earlier than the time point at which the test sample is taken. Recombinant NS1 of influenza B can be used as a positive control.

Western blots show that NS1 levels in biological samples are sufficient to allow detection of these antigens in a variety of different possible immunoassay formats. However, should the levels of NS1 in a particular biological sample prove to be limiting for detection in a particular immunoassay format, then, the live virus in a biological sample can be amplified by infecting cells in vitro, i.e., the NS1 protein in the virus-amplified sample should be detectable in about 6 hr to about 12 hr. The yield of NS1 antigen in biological samples and virus-amplified samples can also be improved by inclusion of protease inhibitors and proteasome inhibitors.

Alternatively, NS1 protein can be detected at the mRNA level. RNA from the sample is reverse transcribed and amplified. Optionally a label is added in the course of the amplification. The amplified nucleic acid is then hybridized with a nucleic acid probe known to be substantially or perfectly complementary to a nucleic acid encoding NS1 from at least one strain of influenza virus B. Hybridiziation is usually detected from the presence of label. Presence of amplified nucleic acid hybridizing to a probe complementary to a nucleic acid encoding influenza B NS1 protein indicates presence of influenza B in the sample. As with antibodies, a probe can be selected to be either panspecific or Nonspecific for different strains of influenza B. The hybridization assay can be performed in an array format. Such a format allows several probes, optionally to nucleic acids encoding NS1 proteins from different strains of influenza B to be included in the array. A probe for the NS1 protein of influenza A or C can also be included, as can probes to other viruses or other pathogens.

### VI. Sample Preparation

Any sample can be used that contains or is thought might contain a detectable concentration of influenza proteins and preferably of NS1. Examples of samples that can be used are lung exudates, cell extracts (respiratory, epithelial lining nose), blood, mucous, and nasal swabs, for example. A high concentration of NS1 can be found in nasal swabs. Thus, a preferred sample for identification of NS1 is nasal secretion.

Binding of NS1 to an antibody occurs in the presence of up to 0.05% SDS, including 0.03% and 0.01%. Therefore, when the nasal or other bodily secretion is not likely to easily be used in a lateral flow format, it can be treated with SDS. Preferably, the amount of SDS added is up to a final concentration of 0.01%, more preferably 0.03% and even more preferably, 0.05%.

### VII. Diagnostic and Therapeutic Kits

Kits are provided for carrying out the present methods. The kits include one or more binding agents, typically antibodies, that specifically bind to NS1 of influenza B. The instant kit optionally contains one or more of the reagents, buffers or additive compositions or reagents disclosed in the examples. The kit can also include a means, such as a device or a system, for removing the influenza viral NS1 from other potential interfering substances in the biological sample. The instant kit can further include, if desired, one or more of various components useful in conducting an assay: e.g., one or more assay containers; one or more control or calibration reagents; one or more solid phase surfaces on which to conduct the assay; or, one or more buffers, additives or detection reagents or antibodies; one or more printed instructions detailing how to use the kit to detect influenza B, e.g. as package inserts and/or container labels, for indicating the quantities of the respective components that are to be used in performing the assay, as well as, guidelines for assessing the results of the assay. The instant kit can contain components useful for conducting a variety of different types of assay formats, including e.g. test strips, sandwich ELISA, Western blot assays, latex agglutination and the like.

### VIII. Antibody Arrays

The invention further provides antibody arrays. Such arrays include a plurality of different antibodies in different regions of the array, each with specificity for NS1 of influenza B. The different antibodies can be selected to have specificity for different strains of influenza B. Antibodies that are panspecific for multiple strains can also be included. Antibodies for influenza A or C NS1 proteins can also be included. Such arrays are useful for detection of influenza B, distinguishing between strains, and distinguishing between influenza A, B and C.

Numerous formats for antibody arrays have been proposed. U.S. Pat. No. 5,922,615 describes a device that utilizes multiple discrete zones of immobilized antibodies on membranes to detect multiple target antigens in an array. U.S. Pat. Nos. 5,458,852, 6,019,944, U.S. Pat. No. 6,143,576 and U.S. patent application Ser. No. 08/902,775 describe diagnostic devices with multiple discrete antibody zones immobilized in a device but not on a membrane for the assay of multiple target antigens. WO 99/67641 describes an array of microspheres is generated with tags that enable the decoding and identification of the specific binders (including antibodies) immobilized on individual microspheres after the microspheres are immobilized on the ends of optical fibers. In U.S. Pat. No. 5,981,180, microspheres are again used to immobilize binders (including antibodies) and the microspheres are distinguished from one another without separating them from the sample by detecting the relative amounts of two different fluorophores that are contained in the microspheres in order to identify the specific binder attached to the microsphere.

All publications and patents cited in this specification are herein incorporated by reference as if each individual publication or patent were specifically and individually indicated to be incorporated by reference. Genbank records referenced by GID or accession number, particularly any polypeptide sequence, polynucleotide sequences or annotation thereof, are incorporated by reference herein. Various changes may be made and equivalents may be substituted without departing from the true spirit and scope of the invention. For example, although the invention has been described primarily for influenza B, a similar strategy can be used mutatis mutandis to detect influenza C. Unless otherwise apparent from the context, any feature, step or embodiment can be used in combination with any other feature, step or embodiment.

### EXAMPLES

### Example 1

### Isolation of Monoclonal Antibodies to NS1 of FLU-B

Monoclonal antibodies were prepared to specifically bind to Flu-B subtype NS1 proteins. The antibodies can be pan specific (i.e., bind to multiple strains of Flu-B) or mono-specific (i.e., bind to one strain of Flu-B without binding to others).

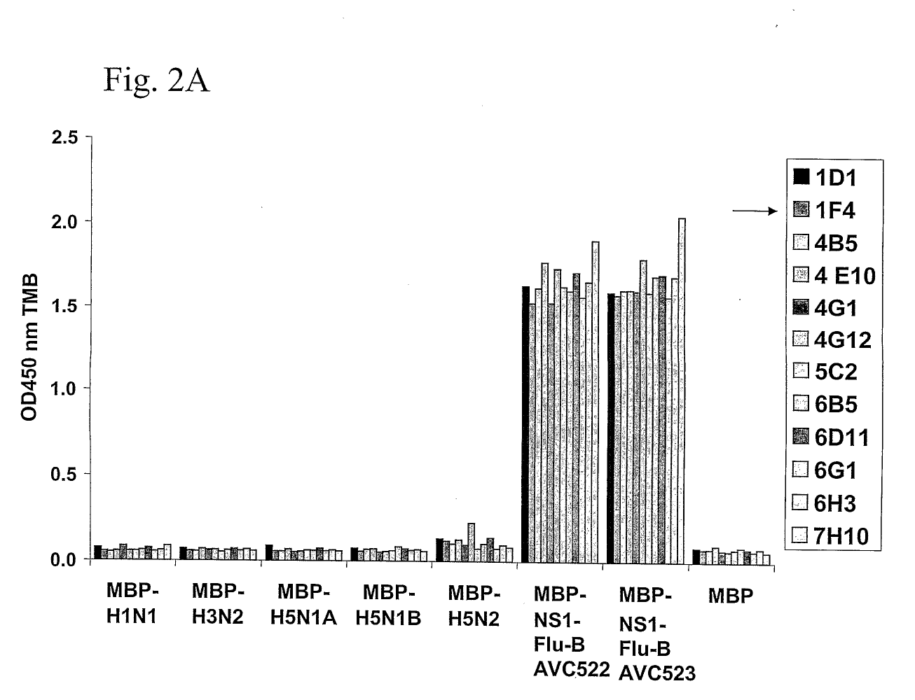

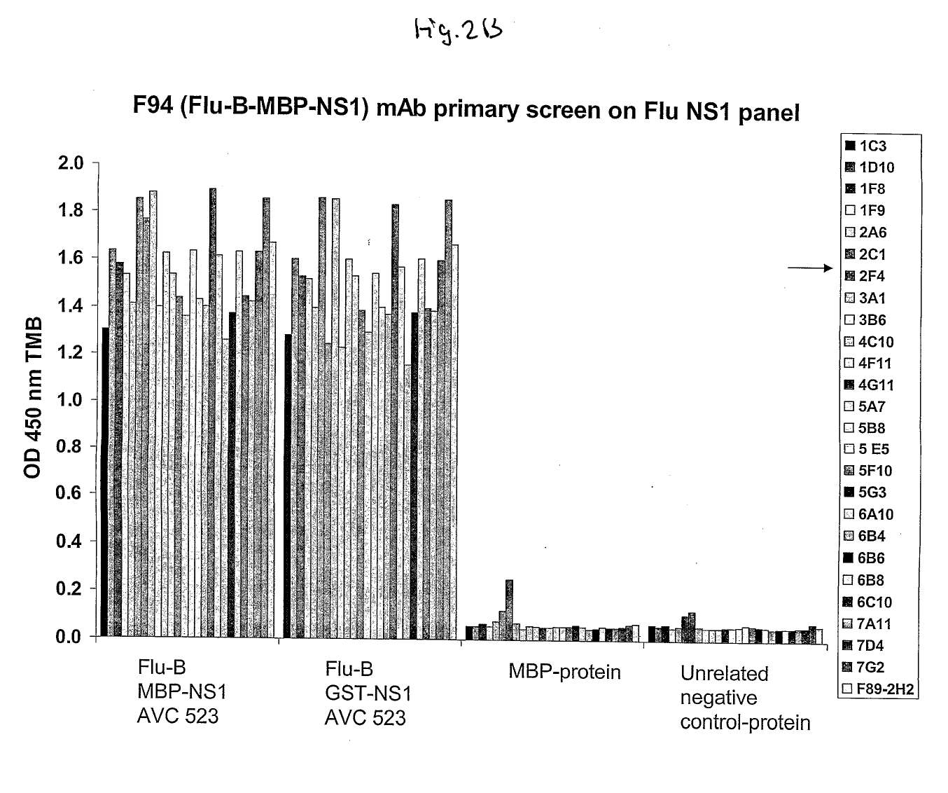

GST and MBP fusion proteins of Flu-B NS1 were generated for the two Flu-B subtypes (B/BA/78; AVC designation: protein # 522; Genbank 325112) and (B/Yagamata/222/2002; AVC designation: protein # 523; Genbank 50300312). The cloning vectors were obtained from Pharmacia (GST) or New England Biolabs (MBP). The NS1 coding regions were synthesized using overlapping oligonucleotides by DNA 2.0 (Menlo Park, Calif.).
1. Mice were immunized with Flu-B MBP-NS1 fusion proteins at doses ranging from 10-100 ug per dose in CFA then IFA and PBS.
2. Splenocytes and lymphocytes were harvested 3 days after the last boost with the corresponding GST-NS1 fusion protein and fused with FOX-NY myeloma cells according to Kohler and Milstein (Nature 1975).
3. The hybridomas were screened first with Flu-B MBP-NS1 in an ELISA (FIGS. 2A and 2B). The positive wells were cloned and rescreened with against both Flu-B NA1 proteins with either MBP and GST tag; mAbs were classified into pan-reactive or subtype reactive.
4. Further screenings were done using lateral flow strips coated with both Flu-B NS1 proteins (#522 and # 523) at different locations on the lateral flow strip to verify reactivity and compatibility for use of the anti Flu-B NS1 mAb on a lateral flow strip. In those assays, detection occurred via a gold labeled anti mouse Ig secondary mAb.
5. Anti Flu-B mAb that showed reactivity with both Flu-B NS1 proteins (AVC #522 and # 523) were tested for capture and detection capacity and for compatibility with each other in a lateral flow sandwich type assay. Candidate capture/selection pairs for both types of Flu-B NS1 proteins were selected and tested for the level of sensitivity for detecting recombinant Flu-B NS1 using the same type of lateral flow sandwich assay.
6. Finally, the antibodies are checked for the ability to detect NS1 in a clinical specimen.

This workflow provides an antibody that will recognize a human clinical specimen.

### Example 2

### Lateral Flow

Using anti-Influenza B NS1 monoclonal antibodies generated according to the above method, a lateral flow test was developed to detect Influenza B NS1. Monoclonal anti-influenza B NS1 antibodies were deposited on an HF075 Millipore membrane at a concentration of ˜0.7 mg/ml using a striper. Some examples of antibodies deposited as capture agents are among the following: F89 1F4, F94 3A1, F89 4D5. A control band was also deposited composed of goat anti-mouse antibody (GAM) also at 1 mg/ml. Flu B NS1 protein was combined with gold conjugated monoclonal anti-NS1 such as F94 3A1 (when F94 3A1 is not used as capture) in 100 ul volume of AVC Flu B buffer. The FluB NS1 proteins used were either recombinant AVC ID 522 (B/BA/78 NS1) and AVC ID 523 (B/YM/222/2002) or clinical samples of from patients known to be infected with influenza B.

The anti-Flu B NS1 antibody striped membrane was inserted into the FluB NS1/anti-NS1 protein solution and flow initiated by capillary action and a wicking pad.

Several combinations of anti-Flu B NS1 capture and detection agents were used in several experiments. The following is an example protocol. The strip tests were run using strips previously striped with goat anti-mouse/F89 1F4 anti-Flu B NS1 monoclonal antibody; 90% M4 viral transport media, 10% of a 10×AVC Flu B buffer; Stocks of NS1 proteins MBP-Flu B NS1 (AVC 522 and AVC 523); gold conjugated F94 3A1 antibody; and Maxisorp ELISA plates. The procedure was performed as follows:
1) Stock NS1 proteins were diluted down in 90% M4 viral transport media, 10% of a 10×AVC Flu B buffer
2.) The stock of NS 1 was diluted down to 0.5 ng/uL by diluting with 90% M4/10% of a 10×AVC Flu B buffer.
3.) Four uL of the gold-conjugated antibody was added to every 100 uL of the buffer
4.) 98 uL of the antibody/buffer mix was added to separate wells in the ELISA plate
5.) 2 uL of the NS1 dilutions were added to the buffer-containing wells (one NS1 per well) to achieve the desired final protein concentration (example 1 ng Flu B NS1)
6.) One well was left with just antibody and buffer to serve as a negative “no NS1” control
7.) The ELISA plate was tapped several times to mix the contents of the wells
8.) The pre-striped strips were added to the wells and observed during development.
   After approximately 15 minutes (when all of the liquid had been absorbed, but the strip was not yet dry) the strips were removed from the wells and scanned into the computer.

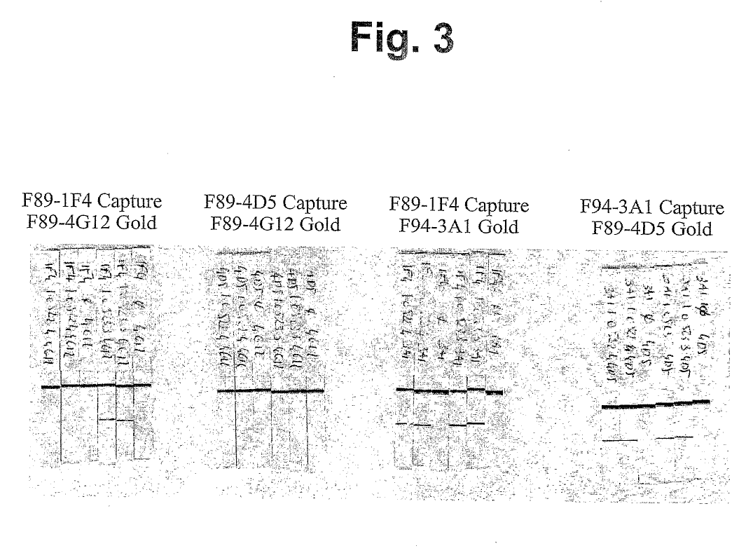

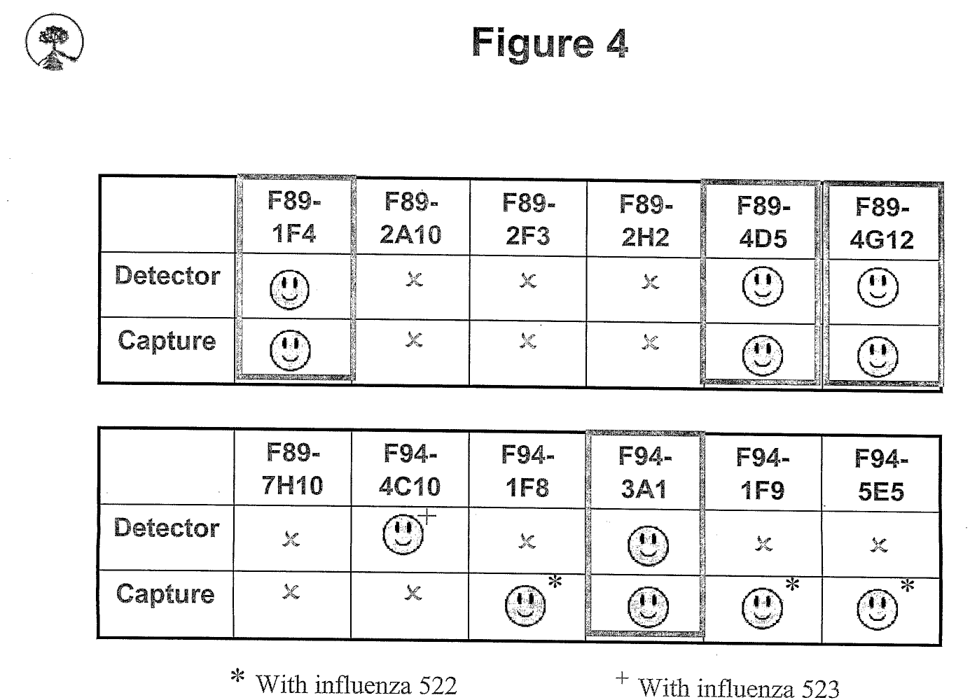

FIG. 3 shows results from testing various pairs of monoclonal antibodies as capture and detection reagent on two strains of influenza B, B/BA78 (also known as strain 522), and B/Yagamata\222\2002, also known as strain 523). The four different panels show four combinations of antibodies. In each panel, tracks 3 and 6 are negative controls. Tracks 1 and 2 are recombinant NS1 from strain 522 and tracks 4 and 5 are recombinant NS1 from strain 523. The presence of additional bands in tracks 4 and 5 but not tracks 1 and 2 of the first panel shows that the F89-F4 capture antibody F89-4G12 detection antibody combination detects the 523 strain but does not detect the 522 strains. The other panels can be analyzed in the same way. The results from this experiment and other similar experiments are summarized in FIG. 4. FIG. 4 shows which antibodies can serve as a capture antibody and which as a detection antibody and whether the antibodies are panspecific for both strains of influenza B (522 and 523) or monospecific to 522 or 523. For example, the F89-1F4 antibody can serve as either a capture or detection antibody and is panspecific. F94-4C10 works as a detection antibody but not as a capture antibody and is specific for influenza B 523. F89-1F4 and F94-3A1 are preferred antibodies for use in lateral flow format.

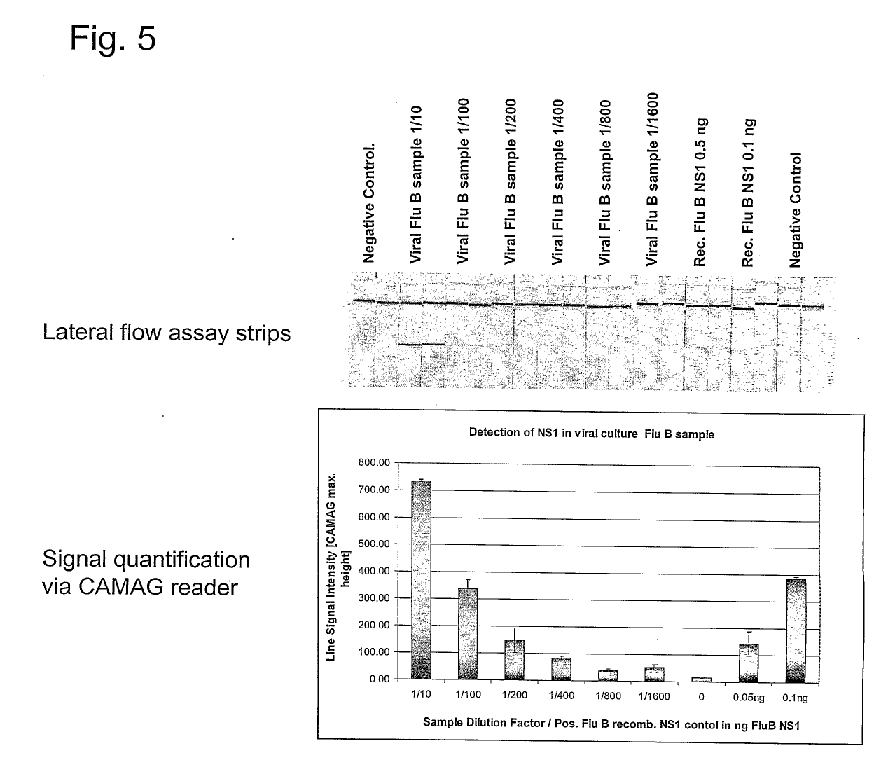

A lateral flow assay was used to identify Influenza B in a patient sample is produced having pan-specific antibodies deposited on the membrane. The patient sample was admixed with a mixture of gold-labeled antibodies that recognize all Influenza B NS1s. The sample was applied to the lateral flow test strip. Presence of influenza B is present a line is shown by a line formed on the strip. FIG. 5 shows the results from different dilutions of a patient sample compared with positive and negative controls. The upper part of the figure shows the actual appearance of lines indicating presence of influenza B. The lower part of the figure indicates the relative intensity of the bands. Influenza B was easily detectable up to a dilution of at least 400 fold.

1. A method for identifying whether a patient is infected with influenza virus type B, comprising:
determining whether NS1 protein of influenza virus type B is present in a patient sample, presence indicating the patient is infected with influenza virus type B.

2. The method of claim 1, wherein the determining comprises
contacting a patient sample with an agent that specifically binds to influenza virus type B protein NS1; and
detecting specific binding between the agent and the NS1 protein, specific binding indicating presence of the influenza virus type B.

3. The method of claim 1, wherein determining comprises determining the presence of mRNA encoding the NS1 protein, and inferring presence of the NS1 protein from the presence of the mRNA.

4. The method of claim 2 wherein the agent is an antibody that specifically binds to the NS1 protein.

5. The method of claim 4, wherein the antibody is panspecific for different strains of influenza type B.

6. The method of claim 4, wherein the antibody is monospecific for a single strain of influenza type B.

7. The method of claim 2, wherein the contacting step comprises, contacting the patient sample with first and second agents that specifically bind to different epitopes of influenza virus type B protein NS1, and the first agent is immobilized on a support, and the detecting step detects a sandwich in which the first and second agents are specifically bound to the NS1 protein to indicate presence of the virus.

8. The method of claim 7, wherein the first and second agents are first and second antibodies.

9. The method of claim 7, wherein the first and/or second agent is a polyclonal antibody.

10. The method of claim 7, wherein the first and/or second agent is panspecific for different strains of influenza type B.

11. The method of claim 1, wherein said patient sample is selected from the group consisting of blood, tissue, a nasal secretion, a lung exudate, a cloacal sample, a fecal sample, a throat swab and saliva.

12. The method of claim 1, further comprising determining whether the sample is infected with influenza virus type A.

13. The method of claim 12, wherein presence or absence of influenza virus type A is determined from presence or absence of influenza virus type A NS1 protein.

14. A kit for the identification and subtyping of influenza virus type B virus in a patient sample, comprising,
an agent that specifically binds to the influenza virus type B NS1 protein, wherein said agent is immobilized on a solid support.

15. The kit of claim 14, wherein said agent is an antibody.

16. The method of claim 2, further comprising quantifying the level of specific binding between the agent and the NS1 protein, the level of specific binding indicating the level of influenza B NS1 protein in the sample.

17. The method of claim 16, wherein the contacting step comprises, contacting the patient sample with first and second agents that specifically bind to different epitopes of influenza virus type B protein NS1, and the first agent is immobilized on a support, and the detecting step detects a sandwich in which the first and second agents are specifically bound to the NS1 protein to indicate presence of the virus.

18. The method of claim 16, wherein the first or second agent comprises an antibody.

19. The method of claim 17, wherein the first and second agents are first and second antibodies.

20. The method of claim 18, wherein the first and/or second agent is panspecific for different strains of influenza type B.


SEQUENCE LISTING (2 sequences)

SEQ ID NO: 1 - protein, 281 aa, Influenza B virus
  misc_feature (107)..(117): Xaa can be any naturally occurring amino acid
MADNMTTTQIEVGPGATNATINFEAGILECYERLSWQRALDYPGQDRLNRLKRKLESRIKTHNKSEPESKRMSLEERKAIGVKMMKVLLFMNPSAGIEGFEPYCMKXXXXXXXXXXXWADYPPTPGKCLDDIEEEPENVDDPTEIVLRDMNNKDARQKIKEEVNTQKEGKFRLTIKRDIRNVLSLRVLVNGTFLKHPNGYKTLSTLHRLNAYDQSGRLVAKLVATDDLTVEDEEDGHRILNSLFERFNEGHSKPIRAAETAVGVLSQFGQEHRLSPEEGDN

SEQ ID NO: 2 - protein, 281 aa, Influenza B virus
MADNMTTTQIEVGPGATNATINFEAGILECYERLSWQRALDYPGQDRLNRLKRKLESRIKTHNKSEPESKRMSLEERKAIGVKMMKVLLFMDPSAGIEGFEPYCMKSSSNSNCPKYNWINYPLTPGRCLDDIEEEPEDVDGPTEIVLRDMNNKDARQKIKEEVNTQKEGKFRLTIKRDIRNVLSLRVLVNGTFLKHPNGYKSLLTLHRLNTYDQSGRLVAKLVATDDLTVEDEEDGHRILNSLFERLNEGHPKPIRAAETAMGVLSQFGQEHRLSPEEGDN